(19) United States
(12) Patent Application Publication
(10) Pub. No.: US 2012/0115199 A1
(43) Pub. Date: May 10, 2012

(54) Enzymatic Hydrolysis Of Pretreated Lignocellulose-Containing Material With Basidiomycota Mycelia And Yeast Cells

(75) Inventors: Xin Li, Raleigh, NC (US); Ye Chen, Cary, NC (US)

(73) Assignee: Novozymes North America, Inc., Franklinton, NC (US)

(21) Appl. No.: 13/063,507

(22) PCT Filed: Sep. 30, 2009
(86) PCT No.: PCT/US09/58912

§ 371 Date: Mar. 11, 2011

Related U.S. Application Data
(60) Provisional application No. 61/101,456, filed on Sep. 30, 2008.

(51) Int. Cl. C12P 7/10

(52) U.S. Cl. 435/165

(57) ABSTRACT

A method for producing a fermentation product from a lignocellulose-containing material comprises pre-treating the lignocellulose-containing material; introducing treated basidiomycota mycelia and/or yeast cells to the pre-treated lignocellulose-containing material; exposing the pre-treated lignocellulose-containing material to an effective amount of hydrolyzing enzyme; and fermenting with a fermenting organism to produce a fermentation product.

### FIELD OF THE INVENTION

Methods for producing fermentation products from lignocellulose-containing material, and more particularly, a method for increasing the efficiency of producing fermentation products from lignocellulose-containing material by treating the material with basidiomycota mycelia and/or yeast cells are disclosed.

### BACKGROUND OF THE INVENTION

Lignocellulose-containing material, or biomass, may be used to produce fermentable sugars, which in turn may be used to produce fermentation products such as renewable fuels and chemicals. Lignocellulose-containing material is a complex structure of cellulose fibers wrapped in a lignin and hemicellulose sheath. Production of fermentation products from lignocellulose-containing material includes pre-treating, hydrolyzing, and fermenting the lignocellulose-containing material.

Conversion of lignocellulose-containing material into renewable fuels and chemicals often involves physical, biological, chemical and/or enzymatic treatment of the biomass with enzymes. In particular, enzymes hydrolyze cellulose to D-glucose, which is a simple fermentable sugar. In lignocellulose-containing material, high doses of enzyme are needed to degrade the cellulose to obtain high yields because lignin and lignin derivatives are believed to inhibit the enzyme(s) from hydrolyzing the cellulose. Such inhibition may occur in at least two ways: the lignin or lignin derivatives preferentially bind with the enzyme thereby preventing the enzyme from binding with or hydrolyzing cellulose, and/or the lignin or lignin derivatives cover portions of the cellulose thereby reducing enzyme access to cellulose. Consequently, when processing lignin-containing biomass, fewer enzymes may be available to degrade cellulose because the lignin or its derivatives may scavenge the enzyme or reduce its activity. Even for the enzymes that are available to degrade cellulose, the available enzyme may not be able to contact the cellulose because lignin may be covering the cellulose. Thus, the effectiveness of the process for digesting cellulose is reduced. In addition, the costs of enzymes are high. Thus, when the amount of enzymes needed to degrade cellulose is high, the processing costs are high and economically unfeasible.

Reduction in the amount of enzyme needed to obtain a satisfactory sugar yield can have a significant impact on process economics. Therefore, improving the efficiency of enzyme use is a major need in the bioconversion process. Several factors are thought to influence enzymatic hydrolysis of cellulose. These factors include lignin content, hemicellulose content, acetyl content, surface area of cellulose and cellulose crystallinity. It is generally understood that the lignin present in complex substrates has a negative effect on enzyme hydrolysis.

The exact role of lignin and lignin derivatives in limiting hydrolysis has been difficult to define. However, it is known that removing the effect of lignin and its derivatives increases hydrolysis of cellulose and increases fermentable sugar yield. This action may open more cellulose surface area for enzymatic attack and may reduce the amount of enzyme that is non-specifically adsorbed on the lignocellulosic substrate. Alternatively, compounds may be used to remove the effect of lignin and its derivatives thereby making cellulose more accessible to enzymatic degradation.

### SUMMARY OF THE INVENTION

Methods for producing fermentation products from lignocellulose-containing material by pre-treating and/or hydrolyzing the material in the presence of treated basidiomycota mycelia and/or yeast cells are disclosed.

Also disclosed are methods for producing a fermentation product from a lignocellulose-containing material including pre-treating the lignocellulose-containing material; introducing treated basidiomycota mycelia and/or yeast cells to the pre-treated lignocellulose-containing material; exposing the pre-treated lignocellulose-containing material to an effective amount of a hydrolyzing enzyme; and fermenting with a fermenting organism to produce a fermentation product. In one aspect, the treated basidiomycota mycelia and/or yeast cells may be introduced to the lignocellulose-containing material prior to exposing the lignocellulose-containing material to an effective amount of a hydrolyzing enzyme. The treated yeast cells may be introduced to the pre-treated lignocellulose-containing material in an amount of about 8% w/w yeast cells/lignocellulose-containing material.

### BRIEF DESCRIPTION OF THE DRAWINGS

FIG. 1. The effects of treated yeast cells on enzyme hydrolysis of PCS.

### DETAILED DESCRIPTION OF THE INVENTION

An improved and more efficient method for enzymatically hydrolyzing lignin-containing biomass by using treated basidiomycota mycelia and/or yeast cells is disclosed.

Lignin is a phenolic polymer that can be derived by the dehydrogenative polymerization of coniferyl alcohol and/or sinapyl alcohol and is found in the cell walls of many plants. As used herein, the term “lignin” refers to the intact structure of the lignin polymer as well as any derivative fragments or compounds of the intact polymer that result from disruption of the lignin structure, including soluble lignin derivatives, condensed lignin and insoluble precipitated lignin. It is believed that lignin and/or lignin derivatives interact with treated basidiomycota mycelia and/or yeast cells in a variety of ways. For example, insoluble precipitated lignin and condensed lignin may have the ability to adsorb treated basidiomycota mycelia and yeast cells from aqueous solutions, and in contrast, soluble lignin derivatives may be adsorbed by treated basidiomycota mycelia and yeast cells.

As used herein, the term “biomass slurry” refers to the aqueous biomass material that undergoes enzymatic hydrolysis. Biomass slurry is produced by mixing biomass, e.g., corn stover, bagasse, etc., with water, buffer, and other pre-treatment materials. The biomass may be pre-treated prior to hydrolysis.

As used herein, the term “lignin blocking” means the reduction or elimination of the deleterious effects of lignin on the process of converting biomass to a fermentation product. In addition, as used herein, the term “effective lignin blocking amount” means any amount useful in facilitating lignin blocking.

In one embodiment, the method utilizes treated basidiomycota mycelia. Without being bound by any particular theory, the treated basidiomycota mycelia may preferentially bind with lignin more readily than cellulose. A lignin-containing biomass slurry may be treated with treated basidiomycota mycelia, for example by introducing treated basidiomycota mycelia directly into the pretreated biomass slurry. It is surmised that the treated basidiomycota mycelia preferentially binds with lignin in the pretreated slurry thereby covering lignin that has precipitated onto the surface of the cellulose, thus impeding the precipitated lignin from binding hydrolyzing enzymes. It is further surmised that treated basidiomycota mycelia may be capable of adsorbing lignin that has not precipitated onto the cellulose surface, allowing cellulose-hydrolyzing enzymes to hydrolyze cellulose more efficiently. Without treatment of the lignin-containing biomass slurry with treated basidiomycota mycelia, lignin may bind a portion of the cellulose-hydrolyzing enzymes rendering them unable to hydrolyze cellulose, or may cover portions of the cellulose, rendering it inaccessible to hydrolyzing enzymes.

In another embodiment, the method utilizes treated yeast cells. Without being bound by any particular theory, the treated yeast cells may preferentially bind with lignin more readily than cellulose. A lignin-containing biomass slurry may be treated with yeast cells, for example by introducing treated yeast cells directly into the pretreated biomass slurry. It is surmised that the treated yeast cells may preferentially bind with lignin in the pretreated slurry thereby covering lignin that has precipitated onto the surface of the cellulose, thus impeding the precipitated lignin from binding hydrolyzing enzymes. It is further surmised that treated yeast cells may be capable of adsorbing lignin that has not precipitated onto the cellulose surface, allowing cellulose-hydrolyzing enzymes to hydrolyze cellulose more efficiently. Without treatment of the lignin-containing biomass slurry with treated yeast cells, lignin may bind a portion of the cellulose-hydrolyzing enzymes rendering them unable to hydrolyze cellulose, or may cover portions of the cellulose, rendering it inaccessible to hydrolyzing enzymes.

Without being bound by any particular theory, it is believed that lignin operates in multiple ways to inhibit enzymes from hydrolyzing cellulose in biomass. Lignin limits the degree to which cellulose can be converted to monomeric sugars by cellulolytic and hemicellulolytic enzymes. The focus of many research activities has been directed to understanding the nature of lignin in cell walls and developing pretreatment processes that are effective in removing it. By understanding the mode in which lignin inhibits enzymatic activity, it is possible to reduce the detrimental effects traditionally caused by lignin content in biomass. As will be described in further detail below, lignocellulose-containing material or biomass may be pretreated prior to being hydrolyzed. For example, pretreatment may take the form of steam pretreatment, alkaline pretreatment, acid pretreatment, or some combination of these. Steam pretreatment physically breaks up the structure of the biomass, i.e., at least partially breaks the bonds connecting the lignin, cellulose, and hemicellulose. Alkaline pretreatment generally includes treatment of the biomass with an alkaline material such as ammonium. Alkaline pretreatment chemically alters the biomass. With respect to the lignin component of the biomass, it is believed that alkaline pretreatment at least partially degrades the lignin forming lignin derivatives and small phenolic fragments that may adversely affect enzyme performance and yeast growth and fermentative capacity. Acid pretreatment also chemically alters the lignin component of the biomass, forming lignin derivatives including condensed lignin that precipitates on the cellulose fiber surface. The condensed lignin may inhibit enzymes from reaching the cellulose by covering the cellulose fiber surface. Other lignin derivatives formed during acid pretreatment include small phenol containing fragments and compounds that may inhibit enzyme function.

It is further believed that treatment of biomass slurry with treated basidiomycota mycelia and/or yeast cells is effective, at least in part, through binding lignin, thus reducing and/or inhibiting non-productive adsorption of cellulose hydrolyzing enzymes to lignin. The treatment of biomass slurry with treated basidiomycota mycelia and/or yeast cells thus improves processing of lignin containing substrates by inhibiting lignin from binding to the enzymes, allowing improved enzyme hydrolysis. Treated basidiomycota mycelia and yeast cells may reduce enzyme load and/or improve performance because the enzymes may not be adversely affected by the lignin thus remain available to more effectively hydrolyze the biomass substrate.

The present method reduces enzyme loading in hydrolysis of lignin containing biomass slurry. The amount of enzyme that is needed to provide hydrolysis is significantly reduced through treating the biomass slurry with treated basidiomycota mycelia and/or yeast cells. Reduction in enzyme loading reduces the overall costs of the biomass conversion processes.

According to one embodiment, the method enhances enzymatic hydrolysis of cellulose using treated basidiomycota mycelia. This method includes the steps of treating a lignin containing biomass slurry with basidiomycota mycelia to provide a treated biomass slurry having a blocked lignin component and exposing the treated biomass slurry to an effective amount of a hydrolyzing enzyme. The basidiomycota mycelia may be added directly to the biomass slurry during or after pretreatment, or before or during hydrolysis. It is preferred that the basidiomycota mycelia be added to the biomass slurry prior to the addition of the cellulose hydrolyzing enzyme and fermenting organism.

According to another embodiment, the method enhances enzymatic hydrolysis of cellulose using treated yeast cells. This method includes the steps of treating a lignin containing biomass slurry with treated yeast cells to provide a treated biomass slurry having a blocked lignin component and exposing the treated biomass slurry to an effective amount of a hydrolyzing enzyme. The treated yeast cells may be added directly to the biomass slurry during or after pretreatment, or before or during hydrolysis. It is preferred that the treated yeast cells be added to the biomass slurry prior to the addition of the cellulose hydrolyzing enzyme and fermenting organism.

### Basidiomycota Mycelia

Basidiomycota is one of two large phyla that comprise the “Higher Fungi” within the Kingdom Fungi. Basidiomycota include mushrooms, puffballs, stinkhorns, bracket fungi, other polypores, jelly fungi, boletes, chanterelles, earth stars, smuts, bunts, rusts, mirror yeasts, and the human pathogenic yeast, Cryptococcus. Generally, basidiomycota are filamentous fungi comprising hyphae (except for those forming yeasts), and reproducing sexually via the formation of specialized club-shaped end cells called basidia that normally bear external spores, which are specialized meiospores called basidiospores.

Mycelium (plural mycelia) is the vegetative part of a fungus, consisting of a mass of branching, thread-like hyphae. Fungal colonies composed of mycelia are found in soil and on or in many other substrates. Typically a single spore germinates into a monokaryotic mycelium which cannot reproduce sexually; when two compatible monokaryotic mycelia join and form a dikaryotic mycelium, that mycelium may form fruiting bodies such as mushrooms. A mycelium may be minute, forming a colony that is too small to see, or it may be extensive:

It is through the mycelium that a fungus absorbs nutrients from its environment. Mycelium is vital in terrestrial and aquatic ecosystems for its role in the decomposition of plant material. It contributes to the organic fraction of soil and its growth releases carbon dioxide back into the atmosphere. The mycelium of mycorrhizal fungi increases the efficiency of water and nutrient absorption of most plants and confers resistance to some plant pathogens. Mycelium is an important food source for many soil invertebrates.

Basidiomycota mycelia for use in the present invention may comprise waste from a fermentation process. More specifically, it may comprise cellulase waste, hemicellulose waste, and lignin modifying enzyme waste from a fermentation process or some combination thereof. Examples of basidiomycota mycelia that may be used include, but are not limited to white and brown rot fungi. Using fermentation waste to improve enzymatic hydrolysis is economically beneficial. It increases the production of fermentation products while at the same time recycling and reusing natural waste from the fermentation process that would otherwise be discarded. The basidiomycota mycelia may also be recovered itself from the fermentation process and be used again to improve enzymatic hydrolysis.

It is contemplated that the basidiomycota mycelia may be treated or processed prior to being introduced to the biomass slurry in order to kill live cells prior to their being introduced to the biomass slurry. If the live cells are not killed, it is possible that they may consume simple sugars resulting from the hydrolysis process thereby reducing the amount of sugar available for fermentation. Treatment or processing may include enzymatic methods, thermal methods, mechanical methods, chemical methods, or a combination of methods. The basidiomycota mycelia may be autoclaved prior to being introduced to the biomass slurry. For example, the basidiomycota mycelia may be autoclaved at 121° C. for 20 minutes.

It is envisioned that first treating biomass slurry with treated basidiomycota mycelia, and then adding the cellulose hydrolyzing enzyme, provides the highest efficiency in cellulose conversion. The treated basidiomycota mycelia treatment of biomass slurry may also occur simultaneously with the addition of a cellulose-hydrolyzing enzyme to the biomass slurry.

Without being bound by any particular theory, it is believed that nonspecific binding of treated basidiomycota mycelia to lignin decreases unproductive binding of enzymes to lignin surfaces or inhibition of enzyme activity due to interactions with lignin. Thus, use of treated basidiomycota mycelia in a process for lignocellulose conversion advantageously facilitates a lowering of the enzyme loading level to achieve the same target conversion percentage.

### Yeast Cells

Yeasts are a growth form of eukaryotic microorganisms classified in the kingdom Fungi. Yeasts are typically unicellular, although some species of yeast may become multicellular through the formation of a string of connected budding cells known as pseudohyphae, or false hyphae as seen in most molds. Yeast size can vary greatly depending on the species, typically measuring 3-4 μm in diameter, although some yeasts can reach over 40 μm.

The yeast species Saccharomyces cerevisiae has been used in baking and fermenting alcoholic beverages for thousands of years. It is also extremely important as a model organism in modern cell biology research, and is the most thoroughly researched eukaryotic microorganism. Other species of yeast, such as Candida albicans, are opportunistic pathogens and can cause infection in humans. Yeasts have recently been used to generate electricity in microbial fuel cells, and produce ethanol from sugar in the fermentation process for the biofuel industry.

Yeasts do not form a specific taxonomic or phylogenetic grouping. The term “yeast” is often taken as a synonym for S. cerevisiae, but the phylogenetic diversity of yeasts is shown by their placement in both divisions Ascomycota and Basidiomycota. Budding yeasts are classified in the order Saccharomycetales.

Yeast cells for use in the present invention may comprise spent yeast cells from a fermentation process. Using spent yeast cells to improve enzymatic hydrolysis is economically beneficial. It increases the production of fermentation products while at the same time recycling and reusing natural waste from the fermentation process that would otherwise be discarded. It is contemplated that the yeast cells may be recovered multiple times from the fermentation process and be used to improve enzymatic hydrolysis.

It is contemplated that the yeast cells may be treated or processed prior to being introduced to the biomass slurry in order to kill live cells prior to their being introduced to the biomass slurry. If the live cells are not killed, it is possible that they may consume simple sugars resulting from the hydrolysis process thereby reducing the amount of sugar available for fermentation. Treatment or processing may include enzymatic methods, thermal methods, mechanical methods, chemical methods, or a combination of methods. The yeast cells may be autoclaved prior to being introduced to the biomass slurry. For example, the yeast cells may be autoclaved at 121° C. for 15 minutes.

It is envisioned that first treating biomass slurry with treated yeast cells, and then adding the cellulose hydrolyzing enzyme, provides the highest efficiency in cellulose conversion. The treated yeast cells treatment of biomass slurry may also occur simultaneously with the addition of a cellulose-hydrolyzing enzyme to the biomass slurry. Treating the biomass slurry with treated yeast cells produces a hydrolysis yield from cellulose that may be measured as percentage improvement in final sugar yield or cellulose conversion rate. By way of example, an approximately 16% improvement in final sugar yield may be obtained in comparison to the hydrolysis yield from cellulose of a biomass slurry that is not treated with treated yeast cells. In addition, by way of further example, an approximately 25% improvement in cellulose conversion rate may be obtained in comparison to hydrolysis yield from cellulose of a biomass slurry that is not treated with treated yeast cells.

Without being bound by any particular theory, it is believed that nonspecific binding of treated yeast cells to lignin decreases unproductive binding of enzymes to lignin surfaces or inhibition of enzyme activity due to interactions with lignin. Thus, use of treated yeast cells in a process for lignocellulose conversion advantageously facilitates a lowering of the enzyme loading level to achieve the same target conversion percentage.

### Lignocellulose-Containing Material

“Lignocellulose” or “lignocellulose-containing material” means material primarily consisting of cellulose, hemicellulose, and lignin. Such material is often referred to as “biomass.”

Biomass is a complex structure of cellulose fibers wrapped in a lignin and hemicellulose sheath. The structure of biomass is such that it is not susceptible to enzymatic hydrolysis. In order to enhance enzymatic hydrolysis, the biomass has to be pre-treated, e.g., by acid hydrolysis under adequate conditions of pressure and temperature, in order to break the lignin seal, saccharify and solubilize the hemicellulose, and disrupt the crystalline structure of the cellulose. The cellulose can then be hydrolyzed enzymatically, e.g., by cellulolytic enzyme treatment, to convert the carbohydrate polymers into fermentable sugars which may be fermented into a desired fermentation product, such as ethanol. Hemicellulolytic enzyme treatments may also be employed to hydrolyze any remaining hemicellulose in the pre-treated biomass.

The biomass may be any material containing lignocellulose. In a preferred embodiment, the biomass contains at least about 30 wt. %, preferably at least about 50 wt. %, more preferably at least about 70 wt. %, even more preferably at least about 90 wt. %, lignocellulose. It is to be understood that the biomass may also comprise other constituents such as proteinaceous material, starch, and sugars such as fermentable or un-fermentable sugars or mixtures thereof.

Biomass is generally found, for example, in the stems, leaves, hulls, husks, and cobs of plants or leaves, branches, and wood of trees. Biomass includes, but is not limited to, herbaceous material, agricultural residues, forestry residues, municipal solid wastes, waste paper, and pulp and paper mill residues. It is to be understood that biomass may be in the form of plant cell wall material containing lignin, cellulose, and hemicellulose in a mixed matrix.

Other examples of suitable biomass include corn fiber, rice straw, pine wood, wood chips, bagasse, paper and pulp processing waste, corn stover, corn cobs, hard wood such as poplar and birch, soft wood, cereal straw such as wheat straw, rice straw, switch grass, Miscanthus, rice hulls, municipal solid waste (MSW), industrial organic waste, office paper, or mixtures thereof.

In a preferred embodiment, the biomass is selected from one or more of corn stover, corn cobs, corn fiber, wheat straw, rice straw, switch grass, and bagasse.

### Pre-Treatment

The biomass may be pre-treated in any suitable way. In accordance with the present invention, pre-treatment may include the introduction of basidiomycota mycelia and/or yeast to the biomass.

Pre-treatment is carried out before hydrolysis or fermentation. The goal of pre-treatment is to separate or release cellulose, hemicellulose, and lignin and thus improving the rate or efficiency of hydrolysis. Pre-treatment methods including wet-oxidation and alkaline pre-treatment target lignin release, while dilute acid treatment and auto-hydrolysis target hemicellulose release. Steam explosion is a pre-treatment method that targets cellulose release.

The pre-treatment step may include a step wherein basidiomycota mycelia and/or yeast cells are added to the biomass. As indicated previously, biomass is typically in the form of biomass slurry when basidiomycota mycelia and/or yeast cells are added. If basidiomycota mycelia and/or yeast cells are added to the biomass slurry during pre-treatment, the remainder of the pre-treatment process remains conventional. However, basidiomycota mycelia and/or yeast cells may alternatively be added during the hydrolysis step such that the pre-treatment step is a conventional pre-treatment step using techniques well known in the art.

Yeast cells may be added in an amount of about 8% w/w yeast cells/lignocellulose-containing material. The biomass may be present during pre-treatment in an amount between about 10-80 wt. %, preferably between about 20-70 wt. %, especially between about 30-60 wt. %, such as around about 50 wt. %.

Chemical, Mechanical and/or Biological Pre-Treatment

The biomass may be pre-treated chemically, mechanically, biologically, or any combination thereof, before or during hydrolysis.

Preferably the chemical, mechanical or biological pre-treatment is carried out prior to the hydrolysis. Alternatively, the chemical, mechanical or biological pre-treatment may be carried out simultaneously with hydrolysis, such as simultaneously with addition of one or more cellulolytic enzymes, or other enzyme activities, to release, e.g., fermentable sugars, such as glucose or maltose.

In one embodiment, the pre-treated biomass may be washed or detoxified in another way. However, washing or detoxification is not required. In a preferred embodiment, the pre-treated biomass is not washed or detoxified.

### Chemical Pre-Treatment

The phrase “chemical pre-treatment” refers to any chemical pre-treatment which promotes the separation or release of cellulose, hemicellulose, or lignin. Examples of suitable chemical pre-treatment methods include treatment with, for example, dilute acid, lime, alkaline, organic solvent, ammonia, sulfur dioxide, or carbon dioxide. Further, wet oxidation and pH-controlled hydrothermolysis are also considered chemical pre-treatment.

In a preferred embodiment, the chemical pre-treatment is acid treatment, more preferably, a continuous dilute or mild acid treatment such as treatment with sulfuric acid, or another organic acid such as acetic acid, citric acid, tartaric acid, succinic acid, hydrogen chloride or mixtures thereof. Other acids may also be used. Mild acid treatment means that the treatment pH lies in the range from about pH 1-5, preferably about pH 1-3. In a specific embodiment the acid concentration is in the range from 0.1 to 2.0 wt. % acid and is preferably sulfuric acid. The acid may be contacted with the biomass and the mixture may be held at a temperature in the range of about 160-220° C., such as about 165-195° C., for periods ranging from minutes to seconds, e.g., 1-60 minutes, such as 2-30 minutes or 3-12 minutes. Addition of strong acids such as sulfuric acid may be applied to remove hemicellulose. The addition of strong acids enhances the digestibility of cellulose.

Other chemical pre-treatment techniques are also contemplated according to the invention. Cellulose solvent treatment has been shown to convert about 90% of cellulose to glucose. It has also been shown that enzymatic hydrolysis could be greatly enhanced when the lignocellulose structure is disrupted. Alkaline H₂O₂, ozone, organosolv (using Lewis acids, FeCl₃, (Al)₂SO₄ in aqueous alcohols), glycerol, dioxane, phenol, or ethylene glycol are among solvents known to disrupt cellulose structure and promote hydrolysis (Mosier et al., 2005, Bioresource Technology 96: 673-686).

Alkaline chemical pre-treatment with base, e.g., NaOH, Na₂CO₃ and ammonia or the like, is also contemplated according to the invention. Pre-treatment methods using ammonia are described in, e.g., WO 2006/110891, WO 2006/11899, WO 2006/11900, WO 2006/110901, which are hereby incorporated by reference.

Wet oxidation techniques involve the use of oxidizing agents such as sulphite based oxidizing agents or the like. Examples of solvent pre-treatments include treatment with DMSO (dimethyl sulfoxide) or the like. Chemical pre-treatment is generally carried out for 1 to 60 minutes, such as from 5 to 30 minutes, but may be carried out for shorter or longer periods of time depending on the material to be pre-treated.

Other examples of suitable pre-treatment methods are described by Schell et al., 2003, Appl. Biochem and Biotechn. Vol. 105-108, p. 69-85, and Mosier et al., 2005, Bioresource Technology 96: 673-686, and U.S. Application Publication No. 2002/0164730, each of which are hereby incorporated by reference.

### Mechanical Pre-Treatment

The phrase “mechanical pre-treatment” refers to any mechanical or physical pre-treatment which promotes the separation or release of cellulose, hemicellulose, or lignin from biomass. For example, mechanical pre-treatment includes various types of milling, irradiation, steaming/steam explosion, and hydrothermolysis.

Mechanical pre-treatment includes comminution, i.e., mechanical reduction of the size. Comminution includes dry milling, wet milling and vibratory ball milling. Mechanical pre-treatment may involve high pressure and/or high temperature (steam explosion). “High pressure” means pressure in the range from about 300 to 600 psi, preferably 400 to 500 psi, such as around 450 psi. High temperature means temperatures in the range from about 100 to 300° C., preferably from about 140 to 235° C. In a preferred embodiment, mechanical pre-treatment is a batch-process, steam gun hydrolyzer system which uses high pressure and high temperature as defined above. A Sunds Hydrolyzer (available from Sunds Defibrator AB (Sweden) may be used for this.

### Combined Chemical and Mechanical Pre-Treatment

In a preferred embodiment, the biomass is pre-treated both chemically and mechanically. For instance, the pre-treatment step may involve dilute or mild acid treatment and high temperature and/or pressure treatment. The chemical and mechanical pre-treatments may be carried out sequentially or simultaneously, as desired.

Accordingly, in a preferred embodiment, the biomass is subjected to both chemical and mechanical pre-treatment to promote the separation or release of cellulose, hemicellulose or lignin.

In a preferred embodiment pre-treatment is carried out as a dilute or mild acid pre-treatment step. In another preferred embodiment pre-treatment is carried out as an ammonia fiber explosion step (or AFEX pre-treatment step).

### Biological Pre-Treatment

The phrase “biological pre-treatment” refers to any biological pre-treatment which promotes the separation or release of cellulose, hemicellulose, or lignin from the biomass. Biological pre-treatment techniques can involve applying lignin-solubilizing microorganisms. See, for example, Hsu, T.-A., 1996, Pretreatment of biomass, in Handbook on Bioethanol: Production and Utilization, Wyman, C. E., ed., Taylor & Francis, Washington, D.C., 179-212; Ghosh, P., and Singh, A., 1993, Physicochemical and biological treatments for enzymatic/microbial conversion of lignocellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, B., 1996, Fermentation of lignocellulosic hydrolyzates for ethanol production, Enz. Microb. Tech. 18: 312-331; and Vallander, L., and Eriksson, K.-E. L., 1990, Production of ethanol from lignocellulosic materials: State of the art, Adv. Biochem. Eng./Biotechnol. 42: 63-95.

### Hydrolysis

Before the pre-treated biomass, preferably in the form of biomass slurry, is fermented it may be hydrolyzed to break down cellulose and hemicellulose into fermentable sugars. In a preferred embodiment, the pre-treated material is hydrolyzed, preferably enzymatically, before fermentation.

The dry solids content during hydrolysis may be in the range from about 5-50 wt. %, preferably about 10-40 wt. %, preferably about 20-30 wt. %. Hydrolysis may in a preferred embodiment be carried out as a fed batch process where the pre-treated biomass (i.e., the substrate) is fed gradually to, e.g., an enzyme containing hydrolysis solution.

In a preferred embodiment hydrolysis is carried out enzymatically. According to the invention, the pre-treated biomass slurry may be hydrolyzed by one or more cellulolytic enzymes, such as cellulases or hemicellulases, or combinations thereof.

In a preferred embodiment, hydrolysis is carried out using a cellulolytic enzyme preparation comprising one or more polypeptides having cellulolytic enhancing activity. In a preferred embodiment, the polypeptide(s) having cellulolytic enhancing activity is of family GH61A origin. Examples of suitable and preferred cellulolytic enzyme preparations and polypeptides having cellulolytic enhancing activity are described in the “Cellulolytic Enzymes” section and “Cellulolytic Enhancing Polypeptides” section below.

As the biomass may contain constituents other than lignin, cellulose and hemicellulose, hydrolysis and/or fermentation may be carried out in the presence of additional enzyme activities such as protease activity, amylase activity, carbohydrate-generating enzyme activity, and esterase activity such as lipase activity.

Enzymatic hydrolysis is preferably carried out in a suitable aqueous environment under conditions which can readily be determined by one skilled in the art. In a preferred embodiment, hydrolysis is carried out at suitable, preferably optimal, conditions for the enzyme(s) in question.

Suitable process time, temperature and pH conditions can readily be determined by one skilled in the art. Preferably, hydrolysis is carried out at a temperature between 25 and 70° C., preferably between 40 and 60° C., especially around 50° C. Hydrolysis is preferably carried out at a pH in the range from pH 3-8, preferably pH 4-6, especially around pH 5. In addition, hydrolysis is typically carried out for between 12 and 192 hours, preferably 16 to 72 hours, more preferably between 24 and 48 hours.

### Fermentation

Fermentable sugars from pre-treated and/or hydrolyzed biomass may be fermented by one or more fermenting organisms capable of fermenting sugars, such as glucose, xylose, mannose, and galactose directly or indirectly into a desired fermentation product. The fermentation conditions depend on the desired fermentation product and fermenting organism and can easily be determined by one of ordinary skill in the art.

Especially in the case of ethanol fermentation, the fermentation may be ongoing for between 1-48 hours, preferably 1-24 hours. In an embodiment, the fermentation is carried out at a temperature between about 20 to 40° C., preferably about 26 to 34° C., in particular around 32° C. In one embodiment, the pH is greater than 5. In another embodiment, the pH is from about pH 3-7, preferably 4-6. However, some, e.g., bacterial fermenting organisms have higher fermentation temperature optima. Therefore, in an embodiment, the fermentation is carried out at temperature between about 40-60° C., such as 50-60° C. The skilled person in the art can easily determine suitable fermentation conditions.

Fermentation can be carried out in a batch, fed-batch, or continuous reactor. Fed-batch fermentation may be fixed volume or variable volume fed-batch. In one embodiment, fed-batch fermentation is employed. The volume and rate of fed-batch fermentation depends on, for example, the fermenting organism, the identity and concentration of fermentable sugars, and the desired fermentation product. Such fermentation rates and volumes can readily be determined by one of ordinary skill in the art.

### SSF, HHF and SHF

Hydrolysis and fermentation may be carried out as a simultaneous hydrolysis and fermentation step (SSF). In general, this means that combined/simultaneous hydrolysis and fermentation are carried out at conditions (e.g., temperature and/or pH) suitable, preferably optimal, for the fermenting organism(s) in question.

The hydrolysis step and fermentation step may be carried out as hybrid hydrolysis and fermentation (HHF). HHF typically begins with a separate partial hydrolysis step and ends with a simultaneous hydrolysis and fermentation step. The separate partial hydrolysis step is an enzymatic cellulose saccharification step typically carried out at conditions (e.g., at higher temperatures) suitable, preferably optimal, for the hydrolyzing enzyme(s) in question. The subsequent simultaneous hydrolysis and fermentation step is typically carried out at conditions suitable for the fermenting organism(s) (often at lower temperatures than the separate hydrolysis step).

The hydrolysis and fermentation steps may also be carried out as separate hydrolysis and fermentation, where the hydrolysis is taken to completion before initiation of fermentation. This is often referred to as “SHF”.

### Recovery

Subsequent to fermentation, the fermentation product may optionally be separated from the fermentation medium in any suitable way. For instance, the medium may be distilled to extract the fermentation product, or the fermentation product may be extracted from the fermentation medium by micro or membrane filtration techniques. Alternatively, the fermentation product may be recovered by stripping. Recovery methods are well known in the art.

### Fermentation Products

The present invention may be used for producing any fermentation product. Preferred fermentation products include alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B12, beta-carotene); and hormones.

Other products include consumable alcohol industry products, e.g., beer and wine; dairy industry products, e.g., fermented dairy products; leather industry products and tobacco industry products. In a preferred embodiment, the fermentation product is an alcohol, especially ethanol. The fermentation product, such as ethanol, obtained according to the invention, may preferably be used as fuel alcohol/ethanol. However, in the case of ethanol, it may also be used as potable ethanol.

### Enzymes

Even if not specifically mentioned in the context of a method or process of the invention, it is to be understood that the enzyme(s) as well as other compounds are used in an effective amount. One or more enzymes may be used.

The phrase “cellulolytic activity” as used herein is understood as comprising enzymes having cellobiohydrolase activity (EC 3.2.1.91), e.g., cellobiohydrolase I and cellobiohydrolase II, as well as endo-glucanase activity (EC 3.2.1.4) and beta-glucosidase activity (EC 3.2.1.21).

The cellulolytic activity may, in a preferred embodiment, be in the form of a preparation of enzymes of fungal origin, such as from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

The cellulolytic enzyme preparation may contain one or more of the following activities: enzyme, hemienzyme, cellulolytic enzyme enhancing activity, beta-glucosidase activity, endoglucanase, cellubiohydrolase, or xylose isomerase.

The enzyme may be a composition as defined in PCT/US2008/065417, which is hereby incorporated by reference. For example, the cellulolytic enzyme preparation comprises a polypeptide having cellulolytic enhancing activity, preferably a family GH61A polypeptide, preferably the one disclosed in WO 2005/074656 (Novozymes). The cellulolytic enzyme preparation may further comprise a beta-glucosidase, such as a beta-glucosidase derived from a strain of the genus Trichoderma, Aspergillus or Penicillium, including the fusion protein having beta-glucosidase activity disclosed in WO 2008/057637. The cellulolytic enzyme preparation may also comprise a CBH II enzyme, preferably Thielavia terrestris cellobiohydrolase II CEL6A. The cellulolytic enzyme preparation may also comprise cellulolytic enzymes, preferably one derived from Trichoderma reesei or Humicola insolens.

The cellulolytic enzyme preparation may also comprising a polypeptide having cellulolytic enhancing activity (GH61A) disclosed in WO 2005/074656; a beta-glucosidase (fusion protein disclosed in WO 2008/057637) and cellulolytic enzymes derived from Trichoderma reesei.

The cellulolytic enzyme may be the commercially available product CELLUCLAST® 1.5L or CELLUZYME™ available from Novozymes A/S, Denmark or ACCELERASE™ 1000 (from Genencor Inc., USA).

A cellulolytic enzyme may be added for hydrolyzing pre-treated biomass slurry. The cellulolytic enzyme may be dosed in the range from 0.1-100 FPU per gram total solids (TS), preferably 0.5-50 FPU per gram TS, especially 1-20 FPU per gram TS. In another embodiment, at least 0.1 mg cellulolytic enzyme per gram total solids (TS), preferably at least 3 mg cellulolytic enzyme per gram TS, such as between 5 and 10 mg cellulolytic enzyme(s) per gram TS is(are) used for hydrolysis.

### Endoglucanase (EG)

One or more endoglucanases may be present during hydrolysis. The term “endoglucanase” means an endo-1,4-(1,3;1,4)-beta-D-glucan 4-glucanohydrolase (E.C. No. 3.2.1.4), which catalyses endo-hydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity may be determined using carboxymethyl cellulose (CMC) hydrolysis according to the procedure of Ghose, 1987, Pure and Appl. Chem. 59: 257-268.

Endoglucanases may be derived from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

### Cellobiohydrolase (CBH)

One or more cellobiohydrollases may be present during hydrolysis. The term “cellobiohydrolase” means a 1,4-beta-D-glucan cellobiohydrolase (E.C. 3.2.1.91), which catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain.

Examples of cellobiohydroloses are mentioned above including CBH I and CBH II from Trichoderma reseei; Humicola insolens and CBH II from Thielavia terrestris cellobiohydrolase (CELL6A).

Cellobiohydrolase activity may be determined according to the procedures described by Lever et al., 1972, Anal. Biochem. 47: 273-279 and by van Tilbeurgh et al., 1982, FEBS Letters 149: 152-156; van Tilbeurgh and Claeyssens, 1985, FEBS Letters 187: 283-288. The Lever et al. method is suitable for assessing hydrolysis of cellulose in corn stover and the method of van Tilbeurgh et al. is suitable for determining the cellobiohydrolase activity on a fluorescent disaccharide derivative.

### Beta-Glucosidase

One or more beta-glucosidases may be present during hydrolysis. The term “beta-glucosidase” means a beta-D-glucoside glucohydrolase (E.C. 3.2.1.21), which catalyzes the hydrolysis of terminal non-reducing beta-D-glucose residues with the release of beta-D-glucose. For purposes of the present invention, beta-glucosidase activity is determined according to the basic procedure described by Venturi et al., 2002, J. Basic Microbiol. 42: 55-66, except different conditions were employed as described herein. One unit of beta-glucosidase activity is defined as 1.0 μmole of p-nitrophenol produced per minute at 50° C., pH 5 from 4 mM p-nitrophenyl-beta-D-glucopyranoside as substrate in 100 mM sodium citrate, 0.01% TWEEN® 20.

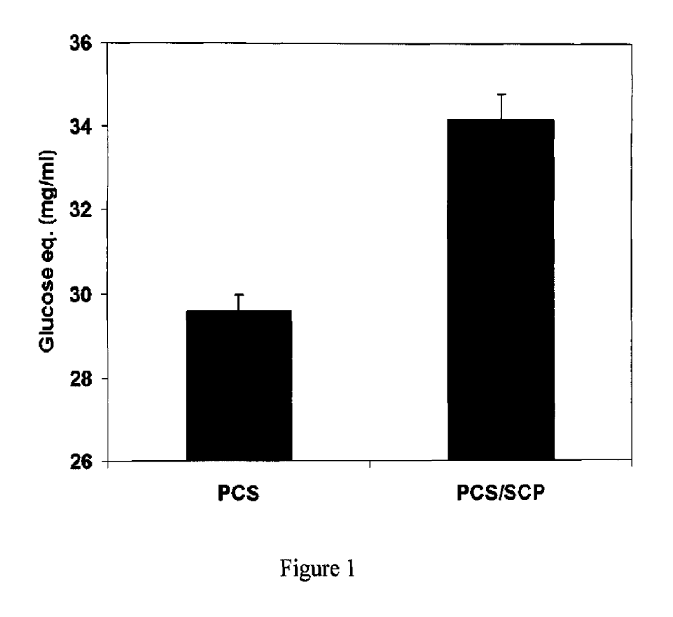

The beta-glucosidase may be of fungal origin, such as a strain of the genus Trichoderma, Aspergillus or Penicillium. The beta-glucosidase may be derived from Trichoderma reesei, such as the beta-glucosidase encoded by the bgl1 gene (see FIG. 1 of EP 562003). The beta-glucosidase may be derived from Aspergillus oryzae (recombinantly produced in Aspergillus oryzae according to WO 2002/095014), Aspergillus fumigatus (recombinantly produced in Aspergillus oryzae according to Example 22 of WO 2002/095014) or Aspergillus niger (1981, J. Appl. Vol 3, pp 157-163).

### Hemicellulase

Hemicellulose can be broken down by hemienzymes and/or acid hydrolysis to release its five and six carbon sugar components. The lignocellulose derived material may be treated with one or more hemicellulases. Any hemicellulase suitable for use in hydrolyzing hemicellulose, preferably into xylose, may be used.

Preferred hemicellulases include xylanases, arabinofuranosidases, acetyl xylan esterase, feruloyl esterase, glucuronidases, endo-galactanase, mannases, endo or exo arabinases, exo-galactanses, and mixtures of two or more thereof. Preferably, the hemicellulase for use in the present invention is an exo-acting hemicellulase, and more preferably, the hemicellulase is an exo-acting hemicellulase which has the ability to hydrolyze hemicellulose under acidic conditions of below pH 7, preferably pH 3-7. An example of hemicellulase suitable for use in the present invention includes VISCOZYME™ (available from Novozymes A/S, Denmark).

The hemicellulase may be a xylanase. The xylanase may preferably be of microbial origin, such as of fungal origin (e.g., Trichoderma, Meripilus, Humicola, Aspergillus, Fusarium) or from a bacterium (e.g., Bacillus). The xylanase may be derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase, more preferably an endo-1,4-beta-xylanase of GH10 or GH11. Examples of commercial xylanases include SHEARZYME™ and BIOFEED WHEAT™ from Novozymes A/S, Denmark.

The hemicellulase may be added in an amount effective to hydrolyze hemicellulose, such as, in amounts from about 0.001 to 0.5 wt. % of total solids (TS), more preferably from about 0.05 to 0.5 wt. % of TS.

Xylanases may be added in amounts of 0.001-1.0 g/kg DM (dry matter) substrate, preferably in the amounts of 0.005-0.5 g/kg DM substrate, and most preferably from 0.05-0.10 g/kg DM substrate.

### Xylose Isomerase

Xylose isomerases (D-xylose ketoisomerase) (E.C. 5.3.1.5.) are enzymes that catalyze the reversible isomerization reaction of D-xylose to D-xylulose. Glucose isomerases convert the reversible isomerization of D-glucose to D-fructose. However, glucose isomarase is sometimes referred to as xylose isomerase.

A xylose isomerase may be used in the method or process and may be any enzyme having xylose isomerase activity and may be derived from any sources, preferably bacterial or fungal origin, such as filamentous fungi or yeast. Examples of bacterial xylose isomerases include the ones belonging to the genera Streptomyces, Actinoplanes, Bacillus and Flavobacterium, and Thermotoga, including T. neapolitana (Vieille et al., 1995, Appl. Environ. Microbiol. 61 (5), 1867-1875) and T. maritime. Examples of fungal xylose isomerases are derived species of Basidiomycetes.

A preferred xylose isomerase is derived from a strain of yeast genus Candida, preferably a strain of Candida boidinii, especially the Candida boidinii xylose isomerase disclosed by, e.g., Vongsuvanlert et al., 1988, Agric. Biol. Chem., 52(7): 1817-1824. The xylose isomerase may preferably be derived from a strain of Candida boidinii (Kloeckera 2201), deposited as DSM 70034 and ATCC 48180, disclosed in Ogata et al., Agric. Biol. Chem., Vol. 33, p. 1519-1520 or Vongsuvanlert et al., 1988, Agric. Biol. Chem., 52(2), p. 1519-1520.

In one embodiment, the xylose isomerase is derived from a strain of Streptomyces, e.g., derived from a strain of Streptomyces murinus (U.S. Pat. No. 4,687,742); S. flavovirens, S. albus, S. achromogenus, S. echinatus, S. wedmorensis all disclosed in U.S. Pat. No. 3,616,221. Other xylose isomerases are disclosed in U.S. Pat. No. 3,622,463, U.S. Pat. No. 4,351,903, U.S. Pat. No. 4,137,126, U.S. Pat. No. 3,625,828, HU patent no. 12,415, DE patent 2,417,642, JP patent no. 69,28,473, and WO 2004/044129, each incorporated by reference herein. The xylose isomerase may be either in immobilized or liquid form. Liquid form is preferred. Examples of commercially available xylose isomerases include SWEETZYME™ T from Novozymes A/S, Denmark. The xylose isomerase is added in an amount to provide an activity level in the range from 0.01-100 IGIU per gram total solids.

### Alpha-Amylase

One or more alpha-amylases may be used. Preferred alpha-amylases are of microbial, such as bacterial or fungal origin. The most suitable alpha-amylase is determined based on process conditions but can easily be done by one skilled in the art.

The preferred alpha-amylase may be an acid alpha-amylase, e.g., fungal acid alpha-amylase or bacterial acid alpha-amylase. The phrase “acid alpha-amylase” means an alpha-amylase (E.C. 3.2.1.1) which added in an effective amount has activity optimum at a pH in the range of 3 to 7, preferably from 3.5 to 6, or more preferably from 4-5.

### Bacterial Alpha-Amylase

As indicated above, the alpha-amylase may be of Bacillus origin. The Bacillus alpha-amylase may preferably be derived from a strain of B. licheniformis, B. amyloliquefaciens, B. subtilis or B. stearothermophilus, but may also be derived from other Bacillus sp. Specific examples of contemplated alpha-amylases include the Bacillus licheniformis alpha-amylase shown in SEQ ID NO: 4 in WO 1999/19467, the Bacillus amyloliquefaciens alpha-amylase SEQ ID NO: 5 in WO 1999/19467 and the Bacillus stearothermophilus alpha-amylase shown in SEQ ID NO: 3 in WO 1999/19467 (all sequences hereby incorporated by reference). In an embodiment, the alpha-amylase may be an enzyme having a degree of identity of at least 60%, preferably at least 70%, more preferred at least 80%, even more preferred at least 90%, such as at least 95%, at least 96%, at least 97%, at least 98% or at least 99% to any of the sequences shown in SEQ ID NO: 1, 2 or 3, respectively, in WO 1999/19467 (hereby incorporated by reference).

The Bacillus alpha-amylase may also be a variant and/or hybrid, especially one described in any of WO 1996/23873, WO 1996/23874, WO 1997/41213, WO 1999/19467, WO 2000/60059, and WO 2002/10355 (all documents hereby incorporated by reference). Specifically contemplated alpha-amylase variants are disclosed in U.S. Pat. Nos. 6,093,562, 6,297,038 or 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta(181-182) compared to the wild-type BSG alpha-amylase amino acid sequence set forth in SEQ ID NO: 3 disclosed in WO 1999/19467 or deletion of amino acids R179 and G180 using SEQ ID NO: 3 in WO 1999/19467 for numbering. Even more preferred are Bacillus alpha-amylases, especially Bacillus stearothermophilus alpha-amylase, which have a double deletion corresponding to delta(181-182) and further comprise a N193F substitution (also denoted 1181*+G182*+N193F) compared to the wild-type BSG alpha-amylase amino acid sequence set forth in SEQ ID NO:3 disclosed in WO 1999/19467.

### Bacterial Hybrid Alpha-Amylase

One or more bacterial hybrid alpha-amylases may be used. A hybrid alpha-amylase specifically contemplated comprises 445 C-terminal amino acid residues of the Bacillus licheniformis alpha-amylase (shown in SEQ ID NO: 4 of WO 1999/19467) and the 37 N-terminal amino acid residues of the alpha-amylase derived from Bacillus amyloliquefaciens (shown in SEQ ID NO: 5 of WO 1999/19467), with one or more, especially all, of the following substitution:

48A+T49I+G107A+H156Y+A181T+N190F+I201F+A209V+Q264S (using the Bacillus licheniformis numbering in SEQ ID NO: 4 of WO 1999/19467). Also preferred are variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using the SEQ ID NO: 5 numbering of WO 1999/19467).

### Fungal Alpha-Amylase

One or more fungal alpha-amylases may be used. Fungal alpha-amylases include alpha-amylases derived from a strain of the genus Aspergillus, such as, Aspergillus oryzae, Aspergillus niger and Aspergillis kawachii alpha-amylases.

A preferred acidic fungal alpha-amylase is a Fungamyl-like alpha-amylase, which is derived from a strain of Aspergillus oryzae. The phrase “Fungamyl-like alpha-amylase” indicates an alpha-amylase which exhibits a high identity, i.e., more than 70%, more than 75%, more than 80%, more than 85% more than 90%, more than 95%, more than 96%, more than 97%, more than 98%, more than 99% or even 100% identity to the mature part of the amino acid sequence shown in SEQ ID NO: 10 in WO 1996/23874.

Another preferred acidic alpha-amylase is derived from a strain Aspergillus niger. The acid fungal alpha-amylase may be the one from A. niger disclosed as “AMYA_ASPNG” in the Swiss-prot/TeEMBL database under the primary accession no. P56271 and described in WO 1989/01969 (Example 3). A commercially available acid fungal alpha-amylase derived from Aspergillus niger is SP288 (available from Novozymes A/S, Denmark).

The fungal alpha-amylase may also be a wild-type enzyme comprising a starch-binding domain (SBD) and an alpha-amylase catalytic domain (i.e., none-hybrid), or a variant thereof. In an embodiment, the wild-type alpha-amylase may be derived from a strain of Aspergillus kawachii.

Other contemplated wild-type alpha-amylases include those derived from a strain of the genera Rhizomucor and Meripilus, preferably a strain of Rhizomucor pusillus (WO 2004/055178 incorporated by reference) or Meripilus giganteus.

The alpha-amylase may be derived from Aspergillus kawachii as disclosed by Kaneko et al., 1996, J. Ferment. Bioeng. 81:292-298, “Molecular-cloning and determination of the nucleotide-sequence of a gene encoding an acid-stable alpha-amylase from Aspergillus kawachii”; and further as EMBL:#AB008370.

### Fungal Hybrid Alpha-Amylase

One or more fungal hybrid alpha-amylases may be used. The fungal acid alpha-amylase may be a hybrid alpha-amylase. Examples of fungal hybrid alpha-amylases include the ones disclosed in WO 2005/003311 or U.S. Application Publication No. 2005/0054071 (Novozymes) or U.S. patent application No. 60/638,614 (Novozymes), which are hereby incorporated by reference. A hybrid alpha-amylase may comprise an alpha-amylase catalytic domain (CD) and a carbohydrate-binding domain/module (CBM), such as a starch binding domain, and optionally a linker.

Specific examples of contemplated hybrid alpha-amylases include those disclosed in Table 1 to 5 of the examples in U.S. patent application No. 60/638,614, including Fungamyl variant with catalytic domain JA118 and Athelia rolfsii SBD (SEQ ID NO:100 in U.S. 60/638,614), Rhizomucor pusillus alpha-amylase with Athelia rolfsii AMG linker and SBD (SEQ ID NO: 101 in U.S. application No. 60/638,614), Rhizomucor pusillus alpha-amylase with Aspergillus niger glucoamylase linker and SBD (which is disclosed in Table 5 as a combination of amino acid sequences SEQ ID NO:20, SEQ ID NO:72 and SEQ ID NO:96 in U.S. application Ser. No. 11/316,535) or as V039 in Table 5 in WO 2006/069290, and Meripilus giganteus alpha-amylase with Athelia rolfsii glucoamylase linker and SBD (SEQ ID NO:102 in U.S. application No. 60/638,614). Other specifically contemplated hybrid alpha-amylases are any of the ones listed in Tables 3, 4, 5, and 6 in Example 4 in U.S. application Ser. No. 11/316,535 and WO 2006/069290, each hereby incorporated by reference.

Other specific examples of contemplated hybrid alpha-amylases include those disclosed in U.S. Application Publication no. 2005/0054071, including those disclosed in Table 3 on page 15, such as Aspergillus niger alpha-amylase with Aspergillus kawachii linker and starch binding domain.

Contemplated are also alpha-amylases which exhibit a high identity to any of above mention alpha-amylases, i.e., more than 70%, more than 75%, more than 80%, more than 85%, more than 90%, more than 95%, more than 96%, more than 97%, more than 98%, more than 99% or even 100% identity to the mature enzyme sequences.

An acid alpha-amylases may according to the invention be added in an amount of 0.1 to 10 AFAU/g DS, preferably 0.10 to 5 AFAU/g DS, especially 0.3 to 2 AFAU/g DS.

### Commercial Alpha-Amylase Products

Preferred commercial compositions comprising alpha-amylase include MYCOLASE from DSM, BAN™, TERMAMYL™ SC, FUNGAMYL™, LIQUOZYME™ X and SAN™ SUPER, SAN™ EXTRA L (Novozymes A/S) and CLARASE™ L-40,000, DEX-LO™, SPEZYME™ FRED, SPEZYME™ AA, and SPEZYME™ DELTA AA (Genencor Int.), and the acid fungal alpha-amylase sold under the trade name SP288 (available from Novozymes A/S, Denmark).

### Carbohydrate-Source Generating Enzyme

The phrase “carbohydrate-source generating enzyme” includes glucoamylase (being glucose generators), beta-amylase and maltogenic amylase (being maltose generators). A carbohydrate-source generating enzyme is capable of producing a carbohydrate that can be used as an energy-source by the fermenting organism(s) in question, for instance, when used in a process for producing a fermentation product such as ethanol. The generated carbohydrate may be converted directly or indirectly to the desired fermentation product, preferably ethanol. A mixture of carbohydrate-source generating enzymes may be present. Especially contemplated mixtures are mixtures of at least a glucoamylase and an alpha-amylase, especially an acid amylase, even more preferred an acid fungal alpha-amylase.

### Glucoamylase

One or more glucoamylases may be used. A glucoamylase may be derived from any suitable source, e.g., derived from a microorganism or a plant. Preferred glucoamylases are of fungal or bacterial origin selected from the group consisting of Aspergillus glucoamylases, in particular A. niger G1 or G2 glucoamylase (Boel et al., 1984, EMBO J. 3 (5), p. 1097-1102), and variants thereof, such as those disclosed in WO 1992/00381, WO 2000/04136 and WO 2001/04273 (from Novozymes, Denmark); the A. awamori glucoamylase disclosed in WO 1984/02921, A. oryzae glucoamylase (Agric. Biol. Chem., 1991, 55 (4), p. 941-949), and variants or fragments thereof. Other Aspergillus glucoamylase variants include variants with enhanced thermal stability: G137A and G139A (Chen et al., 1996, Prot. Eng. 9, 499-505); D257E and D293E/Q (Chen et al., 1995, Prot. Eng. 8, 575-582); N182 (Chen et al., 1994, Biochem. J. 301, 275-281); disulphide bonds, A246C (Fierobe et al., 1996, Biochemistry, 35, 8698-8704; and introduction of Pro residues in position A435 and S436 (Li et al., 1997, Protein Eng. 10, 1199-1204.

Other glucoamylases include Athelia rolfsii (previously denoted Corticium rolfsii), glucoamylase (see U.S. Pat. No. 4,727,026 and Nagasaka et al., 1998, “Purification and properties of the raw-starch-degrading glucoamylases from Corticium rolfsii, Appl Microbiol Biotechnol 50:323-330), and Talaromyces glucoamylases, in particular derived from Talaromyces emersonii (WO 1999/28448), Talaromyces leycettanus (U.S. Pat. No. Re. 32,153), Talaromyces duponti, and Talaromyces thermophilus (U.S. Pat. No. 4,587,215).

Bacterial glucoamylases contemplated include glucoamylases from the genus Clostridium, in particular C. thermoamylolyticum (EP 135,138) and C. thermohydrosulfuricum (WO 1986/01831), and Trametes cingulata disclosed in WO 2006/069289 (which is hereby incorporated by reference).

Hybrid glucoamylases are also contemplated. Examples of the hybrid glucoamylases are disclosed in WO 2005/045018. Specific examples include the hybrid glucoamylase disclosed in Table 1 and 4 of Example 1 of WO 2005/045018, which is hereby incorporated by reference, to the extent it teaches hybrid glucoamylases.

Contemplated are also glucoamylases that exhibit a high identity to any of the above mentioned glucoamylases, i.e., more than 70%, more than 75%, more than 80%, more than 85% more than 90%, more than 95%, more than 96%, more than 97%, more than 98%, more than 99% or even 100% identity to the mature enzymes sequences.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may be added in an amount of 0.02-20 AGU/g DS, preferably 0.1-10 AGU/g DS, especially between 1-5 AGU/g DS, such as 0.5 AGU/g DS.

### Beta-Amylase

One or more beta-amylases may be used. The term “beta-amylase” (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (W. M. Fogarty and C. T. Kelly, Progress in Industrial Microbiology, vol. 15, pp. 112-115, 1979). These beta-amylases are characterized by having optimum temperatures in the range from 40° C. to 65° C. and optimum pH in the range from 4.5 to 7. A commercially available beta-amylase from barley is NOVOZYM™ WBA from Novozymes A/S, Denmark and SPEZYME™ BBA 1500 from Genencor Int., USA.

### Maltogenic Amylase

One or more maltogenic amylases may be used. The amylase may also be a maltogenic alpha-amylase. A maltogenic alpha-amylase (glucan 1,4-alpha-maltohydrolase, E.C. 3.2.1.133) is able to hydrolyze amylose and amylopectin to maltose in the alpha-configuration. A maltogenic amylase from Bacillus stearothermophilus strain NCIB 11837 is commercially available from Novozymes A/S. Maltogenic alpha-amylases are described in U.S. Pat. Nos. 4,598,048, 4,604,355 and 6,162,628, which are hereby incorporated by reference. The maltogenic amylase may be added in an amount of 0.05-5 mg total protein/gram DS or 0.05-5 MANU/g DS.

### Proteases

A protease may be added during hydrolysis, fermentation or simultaneous hydrolysis and fermentation. The protease may be added to deflocculate the fermenting organism, especially yeast, during fermentation. The protease may be any protease. In a preferred embodiment, the protease is an acid protease of microbial origin, preferably of fungal or bacterial origin. An acid fungal protease is preferred, but also other proteases can be used.

Suitable proteases include microbial proteases, such as fungal and bacterial proteases. Preferred proteases are acidic proteases, i.e., proteases characterized by the ability to hydrolyze proteins under acidic conditions below pH 7.

Contemplated acid fungal proteases include fungal proteases derived from Aspergillus, Mucor, Rhizopus, Candida, Coriolus, Endothia, Enthomophtra, Irpex, Penicillium, Sclerotium and Torulopsis. Especially contemplated are proteases derived from Aspergillus niger (see, e.g., Koaze et al., 1964, Agr. Biol. Chem. Japan, 28, 216), Aspergillus saitoi (see, e.g., Yoshida, 1954, J. Agr. Chem. Soc. Japan, 28, 66), Aspergillus awamori (Hayashida et al., 1977, Agric. Biol. Chem., 42(5), 927-933, Aspergillus aculeatus (WO 1995/02044), or Aspergillus oryzae, such as the pepA protease; and acidic proteases from Mucor pusillus or Mucor miehei.

Also contemplated are neutral or alkaline proteases, such as a protease derived from a strain of Bacillus. For example, protease contemplated for the invention is derived from Bacillus amyloliquefaciens and has the sequence obtainable at Swissprot as Accession No. P06832. Also contemplated are the proteases having at least 90% identity to amino acid sequence obtainable at Swissprot as Accession No. P06832 such as at least 92%, at least 95%, at least 96%, at least 97%, at least 98%, or particularly at least 99% identity.

Further contemplated are the proteases having at least 90% identity to amino acid sequence disclosed as SEQ ID NO:1 in WO 2003/048353 such as at 92%, at least 95%, at least 96%, at least 97%, at least 98%, or particularly at least 99% identity.

Also contemplated are papain-like proteases such as proteases within E.C. 3.4.22.* (cysteine protease), such as EC 3.4.22.2 (main), EC 3.4.22.6 (chymopapain), EC 3.4.22.7 (asclepain), EC 3.4.22.14 (actimidain), EC 3.4.22.15 (cathepsin L), EC 3.4.22.25 (glycyl endopeptidase) and EC 3.4.22.30 (caricain).

In an embodiment, the protease may be a protease preparation derived from a strain of Aspergillus, such as Aspergillus oryzae. In another embodiment, the protease may be derived from a strain of Rhizomucor, preferably Rhizomucor meihei. In another contemplated embodiment, the protease may be a protease preparation, preferably a mixture of a proteolytic preparation derived from a strain of Aspergillus, such as Aspergillus oryzae, and a protease derived from a strain of Rhizomucor, preferably Rhizomucor meihei.

Aspartic acid proteases are described in, for example, Hand-book of Proteolytic Enzymes, Edited by A. J. Barrett, N. D. Rawlings and J. F. Woessner, Aca-demic Press, San Diego, 1998, Chapter 270). Suitable examples of aspartic acid protease include, e.g., those disclosed in R. M. Berka et al., Gene, 96, 313 (1990)); (R. M. Berka et al., Gene, 125, 195-198 (1993)); and Gomi et al., Biosci. Biotech. Biochem. 57, 1095-1100 (1993), which are hereby incorporated by reference.

Commercially available products include ALCALASE®, ESPERASE™, FLAVOURZYME™, PROMIX™, NEUTRASE®, RENNILASE®, NOVOZYM™ FM 2.0L, and NOVOZYM™ 50006 (available from Novozymes A/S, Denmark) and GC106™ and SPEZYME™ FAN from Genencor Int., Inc., USA.

The protease may be present in an amount of 0.0001-1 mg enzyme protein per g DS, preferably 0.001 to 0.1 mg enzyme protein per g DS. Alternatively, the protease may be present in an amount of 0.0001 to 1 LAPU/g DS, preferably 0.001 to 0.1 LAPU/g DS and/or 0.0001 to 1 mAU-RH/g DS, preferably 0.001 to 0.1 mAU-RH/g DS.

The invention described and claimed herein is not to be limited in scope by the specific embodiments herein disclosed, since these embodiments are intended as illustrations of several aspects of the invention. Any equivalent embodiments are intended to be within the scope of this invention as well as combinations of one or more of the embodiments. Various modifications of the invention in addition to those shown and described herein will become apparent to those skilled in the art from the foregoing description. Such modifications are also intended to fall within the scope of the appended claims.

Various references are cited herein, the disclosures of which are incorporated by reference in their entireties. The present invention is further described by the following examples which should not be construed as limiting the scope of the invention.

### Materials and Methods

### Identity

The relatedness between two amino acid sequences or between two polynucleotide sequences is described by the parameter “identity”.

For purposes of the present invention, the degree of identity between two amino acid sequences is determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=1, gap penalty=3, windows=5, and diagonals=5.

For purposes of the present invention, the degree of identity between two polynucleotide sequences is determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

### Protease Assays

### AZCL-Casein Assay

A solution of 0.2% of the blue substrate AZCL-casein is suspended in Borax/NaH₂PO₄ buffer pH9 while stirring. The solution is distributed while stirring to microtiter plate (100 microL to each well), 30 microL enzyme sample is added and the plates are incubated in an Eppendorf Thermomixer for 30 minutes at 45° C. and 600 rpm. Denatured enzyme sample (100° C. boiling for 20 min) is used as a blank. After incubation the reaction is stopped by transferring the microtiter plate onto ice and the coloured solution is separated from the solid by centrifugation at 3000 rpm for 5 minutes at 4° C. 60 microL of supernatant is transferred to a microtiter plate and the absorbance at 595 nm is measured using a BioRad Microplate Reader.

pNA-Assay

50 microL protease-containing sample is added to a microtiter plate and the assay is started by adding 100 microL 1 mM pNA substrate (5 mg dissolved in 100 microL DMSO and further diluted to 10 mL with Borax/NaH₂PO₄ buffer pH9.0). The increase in OD₄₀₅ at room temperature is monitored as a measure of the protease activity.

### Glucoamylase Activity (AGU)

Glucoamylase activity may be measured in Glucoamylase Units (AGU).

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

AMG incubation:

| Substrate: | maltose 23.2 mM | |
|---|---|---|
| Buffer: | acetate 0.1M | |
| pH: | 4.30 ± 0.05 | |
| Incubation temperature: | 37° C. ± 1 | |
| Reaction time: | 5 | minutes |
| Enzyme working range: | 0.5-4.0 | AGU/mL |

Color reaction:

| GlucDH: | 430 | U/L |
|---|---|---|
| Mutarotase: | 9 | U/L |
| NAD: | 0.21 | mM |
| Buffer: | phosphate 0.12M; 0.15M NaCl | |
| pH: | 7.60 ± 0.05 | |
| Incubation temperature: | 37° C. ± 1 | |
| Reaction time: | 5 | minutes |
| Wavelength: | 340 | nm |

A folder (EB-SM-0131.02/01) describing this analytical method in more detail is available on request from Novozymes A/S, Denmark, which folder is hereby included by reference.

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

### Acid Alpha-Amylase Activity (AFAU)

When used according to the present invention the activity of an acid alpha-amylase may be measured in AFAU (Acid Fungal Alpha-amylase Units). Alternatively, activity of acid alpha-amylase may be measured in AAU (Acid Alpha-amylase Units).

### Acid Alpha-Amylase Units (AAU)

The acid alpha-amylase activity can be measured in AAU (Acid Alpha-amylase Units), which is an absolute method. One Acid Amylase Unit (AAU) is the quantity of enzyme converting 1 g of starch (100% of dry matter) per hour under standardized conditions into a product having a transmission at 620 nm after reaction with an iodine solution of known strength equal to the one of a color reference.

### Standard Conditions/Reaction Conditions:

Substrate: Soluble starch. Concentration approx. 20 g DS/L.
Buffer: Citrate, approx. 0.13 M, pH=4.2
Iodine solution: 40.176 g potassium iodide+0.088 g iodine/L
City water 15°-20° dH (German degree hardness)
pH: 4.2
Incubation temperature: 30° C.
Reaction time: 11 minutes

### Wavelength: 620 nm

Enzyme concentration: 0.13-0.19 AAU/mL
Enzyme working range: 0.13-0.19 AAU/mL

The starch should be Lintner starch, which is a thin-boiling starch used in the laboratory as colorimetric indicator. Lintner starch is obtained by dilute hydrochloric acid treatment of native starch so that it retains the ability to color blue with iodine. Further details can be found in EP 0140,410 B2, which disclosure is hereby included by reference. Determination of FAU-F

FAU-F Fungal Alpha-Amylase Units (Fungamyl) is measured relative to an enzyme standard of a declared strength.

Reaction conditions

| Temperature | 37° | C. |
|---|---|---|
| pH | 7.15 | |
| Wavelength | 405 | nm |
| Reaction time | 5 | min |
| Measuring time | 2 | min |

A folder (EB-SM-0216.02) describing this standard method in more detail is available on request from Novozymes A/S, Denmark, which folder is hereby included by reference.

### Acid Alpha-Amylase Activity (AFAU)

Acid alpha-amylase activity may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, 10E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

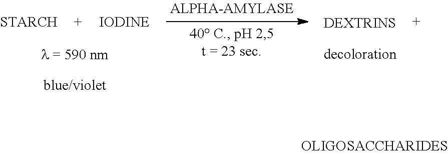

### Standard Conditions/Reaction Conditions:

Substrate: Soluble starch, approx. 0.17 g/L
Buffer: Citrate, approx. 0.03 M
Iodine (I2): 0.03 g/L
CaCl₂: 1.85 mM
pH: 2.50±0.05
Incubation temperature: 40° C.
Reaction time: 23 seconds
Wavelength: 590 nm
Enzyme concentration: 0.025 AFAU/mL
Enzyme working range: 0.01-0.04 AFAU/mL

A folder EB-SM-0259.02/01 describing this analytical method in more detail is available upon request to Novozymes A/S, Denmark, which folder is hereby included by reference.

### Measurement of Cellulase Activity Using Filter Paper Assay (FPU Assay)

### 1. Source of Method

1.1 The method is disclosed in a document entitled “Measurement of Cellulase Activities” by Adney, B. and Baker, J., 1996, Laboratory Analytical Procedure, LAP-006, National Renewable Energy Laboratory (NREL). It is based on the IUPAC method for measuring cellulase activity (Ghose, T. K., 1987, Measurement of Cellulase Activities, Pure & Appl. Chem. 59: 257-268.

### 2. Procedure

2.1 The method is carried out as described by Adney and Baker, 1996, supra, except for the use of a 96 well plates to read the absorbance values after color development, as described below.

### 2.2 Enzyme Assay Tubes:

A rolled filter paper strip (#1 Whatman; 1×6 cm; 50 mg) is added to the bottom of a test tube (13×100 mm).
To the tube is added 1.0 mL of 0.05 M Na-citrate buffer (pH 4.80).
The tubes containing filter paper and buffer are incubated 5 min. at 50° C. (±0.1° C.) in a circulating water bath.
Following incubation, 0.5 mL of enzyme dilution in citrate buffer is added to the tube. Enzyme dilutions are designed to produce values slightly above and below the target value of 2.0 mg glucose.
The tube contents are mixed by gently vortexing for 3 seconds.
2.2.1 After vortexing, the tubes are incubated for 60 mins. at 50° C. (±0.1° C.) in a circulating water bath.
Immediately following the 60 min. incubation, the tubes are removed from the water bath, and 3.0 mL of DNS reagent is added to each tube to stop the reaction. The tubes are vortexed 3 seconds to mix.

### 2.3 Blank and Controls

A reagent blank is prepared by adding 1.5 mL of citrate buffer to a test tube.
A substrate control is prepared by placing a rolled filter paper strip into the bottom of a test tube, and adding 1.5 mL of citrate buffer.
Enzyme controls are prepared for each enzyme dilution by mixing 1.0 mL of citrate buffer with 0.5 mL of the appropriate enzyme dilution.
The reagent blank, substrate control, and enzyme controls are assayed in the same manner as the enzyme assay tubes, and done along with them.

### 2.3 Glucose Standards

A 100 mL stock solution of glucose (10.0 mg/mL) is prepared, and 5 mL aliquots are frozen. Prior to use, aliquots are thawed and vortexed to mix.
Dilutions of the stock solution are made in citrate buffer as follows:
G1=1.0 mL stock+0.5 mL buffer=6.7 mg/mL=3.3 mg/0.5 mL
G2=0.75 mL stock+0.75 mL buffer=5.0 mg/mL=2.5 mg/0.5 mL
G3=0.5 mL stock+1.0 mL buffer=3.3 mg/mL=1.7 mg/0.5 mL
G4=0.2 mL stock+0.8 mL buffer=2.0 mg/mL=1.0 mg/0.5 mL
Glucose standard tubes are prepared by adding 0.5 mL of each dilution to 1.0 mL of citrate buffer.
The glucose standard tubes are assayed in the same manner as the enzyme assay tubes, and done along with them.

### 2.4 Color Development

Following the 60 min. incubation and addition of DNS, the tubes are all boiled together for 5 mins. in a water bath.
After boiling, they are immediately cooled in an ice/water bath.
When cool, the tubes are briefly vortexed, and the pulp is allowed to settle. Then each tube is diluted by adding 50 microL from the tube to 200 microL of ddH₂O in a 96-well plate. Each well is mixed, and the absorbance is read at 540 nm.

### 2.5 Calculations (Examples are Given in the NREL Document)

A glucose standard curve is prepared by graphing glucose concentration (mg/0.5 mL) for the four standards (G1-G4) vs. A₅₄₀. This is fitted using a linear regression (Prism Software), and the equation for the line is used to determine the glucose produced for each of the enzyme assay tubes.
A plot of glucose produced (mg/0.5 mL) vs. total enzyme dilution is prepared, with the Y-axis (enzyme dilution) being on a log scale.
A line is drawn between the enzyme dilution that produced just above 2.0 mg glucose and the dilution that produced just below that. From this line, it is determined the enzyme dilution that would have produced exactly 2.0 mg of glucose.
The Filter Paper Units/mL (FPU/mL) are calculated as follows:

FPU/mL=0.37/enzyme dilution producing 2.0 mg glucose.

### EXAMPLES

The effect of adding yeast cells on sugar yield was tested. Yeast cells were added to washed pre-treated corn stover (PCS) slurry prior to hydrolysis and varying kinds of enzymes were added during hydrolysis. The sugar content was measured at 72 hours after the start of hydrolysis.

Cellulase preparation A: Cellulase preparation A is a cellulolytic composition comprising a polypeptide having cellulolytic enhancing activity (GH61A) disclosed in WO 2005/074656; a beta-glucosidase (a fusion protein disclosed in WO 2008/057637); and cellulolytic enzymes preparation derived from Trichoderma reesei. Cellulase preparation A is disclosed in co-pending international application no. PCT/US2008/065417.

Yeast cell samples were obtained from the ethanol fermentation industry. Cellulase preparation A was used for hydrolysis. The yeast cells were autoclaved at 121° C. for 15 minutes. The pre-processed yeast cells were added to washed pre-treated corn stover (PCS) slurry and mixed. The mixture was hydrolyzed by Cellulase preparation A in an amount of 6.0 mg enzyme protein/g total solids at 50° C. for 72 hours.

The content of released sugar was determined by PHBA method and confirmed by HPLC (High Pressure Liquid Chromatography). As shown in FIG. 1, the addition of treated yeast cells to the enzymatic hydrolysis process increased the final sugar yield. When 8% w/w yeast cells/lignocellulose-containing material was added to PCS slurry before hydrolysis, the sugar yield increased from 29.5 g/L to 34.2 g/L, and the carbohydrate conversion rate improved from 72% to 90.4%.

1-19. (canceled)

20. A method for producing a fermentation product from a lignocellulose-containing material, comprising:
(a) pre-treating the lignocellulose-containing material;
(b) introducing basidiomycota mycelia to the pre-treated lignocellulose-containing material;
(c) exposing the pre-treated lignocellulose-containing material to an effective amount of a hydrolyzing enzyme; and
(d) fermenting with a fermenting organism to produce a fermentation product.

21. The method of claim 20, wherein the basidiomycota mycelia is introduced to the lignocellulose-containing material prior to exposing the lignocellulose-containing material to an effective amount of a hydrolyzing enzyme.

22. The method of claim 20, wherein the basidiomycota mycelia is introduced to the lignocellulose-containing material at the same time as exposing the lignocellulose-containing material to an effective amount of a hydrolyzing enzyme.

23. The method of claim 20, wherein the basidiomycota mycelia are treated prior to being introduced to the pre-treated lignocellulose-containing material.

24. The method of claim 23, wherein the basidiomycota mycelia are autoclaved.

25. The method of claim 20, wherein the lignocellulose-containing material is pre-treated using acid pre-treatment.

26. The method of claim 20, wherein the lignocellulose-containing material is selected from the group consisting of bagasse, corn cobs, corn fiber, corn stover, rice straw, switch grass, wheat straw, and combinations thereof.

27. The method of claim 20, wherein the fermentation product is ethanol.

28. A method for enhancing enzymatic hydrolysis of a lignocellulose-containing material, comprising:
(a) introducing an effective lignin blocking amount of basidiomycota mycelia to the lignocellulose-containing material, and
(b) exposing the lignocellulose-containing material to an effective amount of a hydrolyzing enzyme.

29. A method for producing a fermentation product from a lignocellulose-containing material, comprising:
(a) pre-treating the lignocellulose-containing material;
(b) introducing yeast cells to the pre-treated lignocellulose-containing material;
(c) exposing the pre-treated lignocellulose-containing material to an effective amount of a hydrolyzing enzyme; and
(d) fermenting with a fermenting organism to produce a fermentation product.

30. The method of claim 29, wherein the yeast cells are introduced to the lignocellulose-containing material prior to exposing the lignocellulose-containing material to an effective amount of a hydrolyzing enzyme.

31. The method of claim 29, wherein the yeast cells are introduced to the lignocellulose-containing material at the same time as exposing the lignocellulose-containing material to an effective amount of a hydrolyzing enzyme.

32. The method of claim 29, wherein the yeast cells are introduced to the lignocellulose-containing material in an amount of about 8% w/w yeast cells/lignocellulose-containing material.

33. The method of claim 29, wherein the yeast cells are treated prior to being introduced to the pre-treated lignocellulose-containing material.

34. The method of claim 33, wherein the yeast cells are autoclaved.

35. The method of claim 29, wherein the lignocellulose-containing material is pre-treated using acid pre-treatment.

36. The method of claim 29, wherein the lignocellulose-containing material is selected from the group consisting of bagasse, corn cobs, corn fiber, corn stover, rice straw, switch grass, wheat straw, and combinations thereof.

37. The method of claim 29, wherein the fermentation product is ethanol.
